(19) United States
(12) Patent Application Publication
(10) Pub. No.: US 2014/0074103 A1
(43) Pub. Date: Mar. 13, 2014

(54) SUBCHONDRAL TREATMENT OF BONE DEFECTS WITH BONE-DERIVED IMPLANT

(71) Applicant: Zimmer GmbH, Inc., Winterthur (CH)

(72) Inventors: Christopher D. MANDEEN, West Chester, PA (US); Shaun B. HANSON, West Chester, PA (US); David L. NICHOLS, West Chester, PA (US)

(73) Assignee: Zimmer GmbH, Inc., Winterthur (CH)

(21) Appl. No.: 14/022,020

(22) Filed: Sep. 9, 2013

Related U.S. Application Data
(60) Provisional application No. 61/697,931, filed on Sep. 7, 2012.

(51) Int. Cl. A61B 17/88

(52) U.S. Cl. CPC A61B 17/8808
USPC 606/93

(57) ABSTRACT

Methods of treating bone defects of the joint, particularly at the subchondral level, such as with bone-derived implants or injectable bone augmentation materials, either in combination or alone, are provided. A system of instruments and tools for delivering the implant and injectable material is also provided. These instruments and tools may be provided with depth control features.

### CROSS-REFERENCE TO RELATED APPLICATIONS

This application claims priority to U.S. Provisional No. 61/697,931 filed Sep. 7, 2012 and entitled “SUBCHONDRAL TREATMENT OF BONE DEFECTS WITH BONE-DERIVED IMPLANT,” the contents of which are incorporated by reference in their entirety.

### FIELD

The present invention relates to methods and devices for the surgical treatment of bone defects, and more particularly to implants and related delivery tools for the surgical repair or treatment of subchondral bone defects, especially at or near a joint, and associated methods. Even more particularly, the implantable device can comprise a bone-derived material.

### BACKGROUND

Human joints, in particular the knee, hip and spine, are susceptible to degeneration from disease, trauma, and long-term repetitive use that eventually lead to pain. Knee pain, for example, is the impetus for a wide majority of medical treatments and associated medical costs. The most popular theory arising from the medical community is that knee pain results from bone-on-bone contact or inadequate cartilage cushioning. These conditions are believed to frequently result from the progression of osteoarthritis, which is measured in terms of narrowing of the joint space. Therefore, the severity of osteoarthritis is believed to be an indicator or precursor to joint pain. Most surgeons and medical practitioners thus base their treatments for pain relief on this theory. For example, the typical treatment is to administer pain medication, or more drastically, to perform some type of joint resurfacing or joint replacement surgery.

However, the severity of osteoarthritis, especially in joints such as the knee and ankle, has been found to correlate poorly with the incidence and magnitude of knee pain. Because of this, surgeons and medical practitioners have struggled to deliver consistent, reliable pain relief to patients especially if preservation of the joint is desired.

Whether by external physical force, disease, or the natural aging process, structural damage to bone can cause injury, trauma, degeneration or erosion of otherwise healthy tissue. The resultant damage can be characterized as a bone defect that can take the form of a fissure, fracture, microfracture, lesion, edema, tumor, or sclerotic hardening, for example. Particularly in joints, the damage may not be limited to a bone defect, and may also include cartilage loss (especially articular cartilage), tendon damage, and inflammation in the surrounding area.

Patients most often seek treatment because of pain and deterioration of quality of life attributed to the osteoarthritis. The goal of surgical and non-surgical treatments for osteoarthritis is to reduce or eliminate pain and restore joint function. Both non-surgical and surgical treatments are currently available for joint repair.

Non-surgical treatments include weight loss (for the overweight patient), activity modification (low impact exercise), quadriceps strengthening, patellar taping, analgesic and anti-inflammatory medications, and with corticosteroid and/or viscosupplements. Typically, non-surgical treatments, usually involving pharmacological intervention such as the administration of non-steroidal anti-inflammatory drugs or injection of hyaluronic acid-based products, are initially administered to patients experiencing relatively less severe pain or joint complications. However, when non-surgical treatments prove ineffective, or for patients with severe pain or bone injury, surgical intervention is often necessary.

Surgical options include arthroscopic partial meniscectomy and loose body removal. Most surgical treatments conventionally employ mechanical fixation devices such as screws, plates, staples, rods, sutures, and the like are commonly used to repair damaged bone. These fixation devices can be implanted at, or around, the damaged region to stabilize or immobilize the weakened area, in order to promote healing and provide support. Injectable or fillable hardening materials such as bone cements, bone void fillers, or bone substitute materials are also commonly used to stabilize bone defects.

High tibial osteotomy (HTO) or total knee arthroplasty (TKA) is often recommended for patients with severe pain associated with osteoarthritis, especially when other non-invasive options have failed. Both procedures have been shown to be effective in treating knee pain associated with osteoarthritis.

However, patients only elect HTO or TKA with reluctance. Both HTO and TKA are major surgical interventions and may be associated with severe complications. HTO is a painful procedure that may require a long recovery. TKA patients often also report the replaced knee lacks a “natural feel” and have functional limitations. Moreover, both HTO and TKA have limited durability. Accordingly, it would be desirable to provide a medical procedure that addresses the pain associated with osteoarthritis and provides an alternative to a HTO or TKA procedure.

It is desirable to provide treatment methods and implants that can provide mechanical strength and structural integrity to the area to be treated, while also being as physiologically and biologically compatible as possible to reduce or eliminate any potential negative effects to the patient. It would also be beneficial to provide such devices having the ability to facilitate the dispersal of hardening or augmentation material in the same area. It is further desirable to provide implants that are configured for the treatment or repair of bone defects particularly at the joints, and even more particularly at the subchondral bone level.

### SUMMARY

The present disclosure provides methods of treating bone defects of the joint, particularly at the subchondral level, such as with implants formed of bone material or injectable materials, either in combination or alone. Also provided are delivery tools for delivering the implants to the area of bone to be treated. These delivery tools may be provided with depth control for more accurate delivery.

In one exemplary embodiment, a system for treating a subchondral defect of a bone of a joint is provided. The system comprises a delivery instrument for injecting a bone augmentation material. The instrument may comprise a fenestrated delivery device configured to attach to an injection system. The delivery device may have a bone cutting edge at its tip to create a bone cavity in the bone. The delivery instrument may also include a protective sleeve configured to receive the fenestrated delivery device.

The system may also include an injection system containing bone augmentation material, and an implant comprising bone derived material. The implant may be configured to plug the bone cavity after injection of the bone augmentation material. The system may also optionally include a guide wire. The implant as well as the delivery device may be fully cannulated to work with the guide wire. In some embodiments, the delivery device may comprise depth control features or components. For example, the delivery device may comprise a collar for depth control. The delivery device may also comprise visual markers for depth control.

Additionally, the system may comprise an impacter instrument to push the implant through the protective sleeve and into the bone cavity. The bone augmentation material may comprise a bone hardening or bone substitute material. In some embodiments, the implant may be an allograft. A navigation guide, template guide, or other imaging tool may also be provided with the system for guiding the instrument, device or implant toward the subchondral defect or bone cavity.

In another embodiment, a method of treating a subchondral defect of a bone is provided. The method comprises the steps of inserting a guide wire into the bone toward the subchondral defect, the guide wire being inserted without violating the articular surface of the bone; inserting a delivery device over the guide wire and into the bone to create a bone cavity at a subchondral area near the subchondral defect; injecting a bone augmentation material into the bone cavity; and inserting an implant into the bone cavity, wherein the method preserves the articular surface of the bone. The subchondral bone defect may comprise a bone marrow lesion or bone marrow edema.

In some embodiments, the wire may be inserted using a navigation guide, template guide, or other imaging tool. The injectable material may comprise a bone substitute or bone hardening material. The method may further include inserting the delivery device through a protective sleeve and into the bone. In addition, the method may include pushing the implant through a protective sleeve into the bone cavity using an impacter instrument.

In yet another method, a method of treating a subchondral defect of a bone is provided that does not require a guide wire. The method comprises the steps of inserting a delivery device through a protective sleeve and toward the subchondral defect to create a bone cavity at a subchondral area near the subchondral defect, the delivery device being inserted without violating the articular surface of the bone; injecting a bone augmentation material into the bone cavity; and inserting an implant into the bone cavity; wherein the method preserves the articular surface of the bone. The subchondral bone defect may comprise a bone marrow lesion or bone marrow edema.

A navigation guide, template guide, or other imaging tool may also be used to guide the instrument, device or implant toward the subchondral defect or bone cavity. In some embodiments, the injectable material may comprise a bone substitute or bone hardening material. In addition, the method may include pushing the implant through a protective sleeve into the bone cavity using an impacter instrument.

It is to be understood that both the foregoing general description and the following detailed description are exemplary and explanatory only and are not restrictive of the disclosure. Additional features of the disclosure will be set forth in part in the description which follows or may be learned by practice of the disclosure.

### BRIEF DESCRIPTION OF THE DRAWINGS

The accompanying drawings, which are incorporated in and constitute a part of this specification, illustrate several embodiments of the disclosure and together with the description, serve to explain the principles of the disclosure.

FIGS. 1, 2A, 2B, and 3-6 illustrate an exemplary embodiment of a method of treating a subchondral bone defect of a joint in which:

FIG. 1 shows a wire inserted through a protective sleeve to the subchondral area to be treated;

FIG. 2A shows another step of treating the bone defect of the joint of FIG. 1 by drilling a delivery pin to the subchondral area to be treated;

FIG. 2B is a perspective view of the delivery pin of FIG. 2A;

FIG. 3 shows yet another step of treating the bone defect of the joint of FIG. 1 by injecting an augmentation material into the subchondral area to be treated with an injection device;

FIG. 4 shows still another step of treating the bone defect of the joint of FIG. 1 by inserting a wire to the subchondral area to be treated;

FIG. 5 shows further still another step of treating the bone defect of the joint of FIG. 1 by inserting a cannulated implant into the subchondral area to be treated with an inserter instrument; and

FIG. 6 shows the cannulated implant inserted in the subchondral area of the joint of FIG. 1.

FIGS. 7A, 7B and 8 illustrate another exemplary embodiment of a method of treating a subchondral bone defect of a joint in which:

FIG. 7A shows a step of treating the bone defect in the subchondral area of the joint with a delivery instrument comprising an injection device and protective sleeve;

FIG. 7B shows an exploded view of the delivery instrument of FIG. 7A; and

FIG. 8 shows an implant inserted in the subchondral area of the joint of FIG. 7A with an inserter instrument and protective sleeve.

### DESCRIPTION OF THE EMBODIMENTS

Methods, devices and instruments for treating joint pain to restore natural joint function and preserving, as much as possible, the joint's articular and cartilage surface are known. Treatments through the joint that violate the articular and cartilage surface often weaken the bone and have unpredictable results. Rather than focusing on treatment of pain through the joint, alternative treatments that diagnose and treat pain at its source in the subchondral region of a bone of a joint to relieve the pain are provided. Pain associated with joints, especially osteoarthritic joints, can be correlated to bone defects or changes at the subchondral level rather than, for example, the severity of osteoarthritic progression or defects at the articular surface level. In particular, bone defects, such as bone marrow lesions, edema, fissures, fractures, hardened bone, etc. near the joint surface lead to a mechanical disadvantage and abnormal stress distribution in the periarticular bone, which may cause inflammation and generate pain. By altering the makeup of the periarticular bone (which may or may not be sclerotic) in relation to the surrounding region, it is possible to change the structural integrity of the affected bone and restore normal healing function, thus leading to a resolution of the inflammation surrounding the defect.

Treatment of the bone by mechanical and biological means to restore the normal physiologic stress distribution, and restore the healing balance of the bone tissue at the subchondral level, is a more effect way of treating pain than conventional techniques. That is, treatment can be effectively achieved by mechanically strengthening or stabilizing the defect, and biologically initiating or stimulating a healing response to the defect. Methods, devices, and systems for a subchondral procedure that achieve these goals are disclosed in co-owned U.S. Pat. No. 8,062,364 entitled “OSTEOARTHRITIS TREATMENT AND DEVICE” as well as in co-owned and co-pending U.S. Patent Application Publication Nos. 2011/0125156 entitled “METHOD FOR TREATING JOINT PAIN AND ASSOCIATED INSTRUMENTS” and 2011/0125157 entitled “SUBCHONDRAL TREATMENT OF JOINT PAIN,” both of which were filed on Nov. 19, 2010, the contents of which are incorporated by reference in their entirety. This subchondral procedure, and its associated devices, instruments, etc. are also marketed under the registered trademark name of SUBCHONDROPLASTY™. The SUBCHONDROPLASTY™ procedure is a response to a desire for an alternative to patients facing partial or total knee replacement.

In general, the SUBCHONDROPLASTY™ or SC™ technique is intended to both strengthen the bone and stimulate the bone. In an SC™ procedure, bone fractures or non-unions are stabilized, integrated or healed, which results in reduction of a bone defect, such as a bone marrow lesion or edema. In addition, the SC™ procedure restores or alters the distribution of forces in a joint to thereby relieve pain. The SC™ procedure can be performed arthroscopically or percutaneously to treat pain by stabilizing chronic stress fracture, resolving any chronic bone marrow lesion or edema, and preserving, as much as possible, the articular surfaces of the joint. The SUBCHONDROPLASTY™ procedure generally comprises evaluating a joint, for example, by taking an image of the joint, detecting the presence of one or more subchondral defects, diagnosing, which of these subchondral defects is the source of pain, and determining an extent of treatment for the subchondral defect. The technique is particularly suited for treating chronic defects or injuries, where the patient's natural healing response has not resolved the defect. It should be noted, however, that the technique is equally applicable to treatment of defects in the subchondral region of bone where the defect is due to an acute injury or from other violations. Several exemplary treatment modalities for the SC™ procedure for the different extents of treatment needed can be employed. Accordingly, a medical practitioner may elect to use the techniques and devices described herein to subchondrally treat any number of bone defects, as he deems appropriate.

Detection and identification of the relevant bone marrow lesion or bone marrow edema (BML or BME) can be achieved by imaging, e.g., magnetic resonance imaging (MRI), X-ray, bone scans, manual palpation, chemical or biological assay, and the like. A T1-weighted MRI can be used to detect sclerotic bone, for example. Another example is that a T2-weighted MRI can be used to detect lesions, edemas, and cysts. X-ray imaging may be suitable for early-stage as well as end-stage arthritis. From the imaging, certain defects may be identified as the source of pain. In general, defects that are associated with chronic injury and chronic deficit of healing are differentiated from defects that result, e.g., from diminished bone density. SC™ treatments are appropriate for a BML or BME that may be characterized as a bone defect that is chronically unable to heal (or remodel) itself, which may cause a non-union of the bone, stress or insufficiency fractures, and perceptible pain. Factors considered may include, among other things, the nature of the defect, size of the defect, location of the defect, etc. For example, bone defects at the edge near the articular surface of periphery of a joint may be often considered eligible for treatment due to edge-loading effects as well as the likelihood of bone hardening at these locations. A bone defect caused by an acute injury would generally be able to heal itself through the patient's own natural healing process. However, in such situations where the bone defect is due to an acute injury and either the defect does not heal on its own, or the medical practitioner decides that the present technique is appropriate, SC™ treatment can be administered on acute stress fractures, BML or BME, or other subchondral defects, as previously mentioned.

The SC™ treatment may continue after surgery. In particular, the patient may be monitored for a change in pain scores, or positive change in function. For example, patients are also checked to see when they are able to perform full weight-bearing activity and when they can return to normal activity. Of note, the SC™ procedure can be revised and thus allows for optional further treatment in the event that a patient requires or desires a joint replacement or other type of procedure. The procedure does not exclude a future joint repair or replacement treatment to be applied, and thus may also be performed in conjunction with other procedures, such as cartilage resurfacing, regeneration or replacement, if desired. In those instances where additional treatment is desired, the SC™ treated area may remain undisturbed while the additional treatment is performed, such as where cartilage resurfacing is desired. Alternatively, the SC™ treated area can be removed, and not create an obstacle to the additional treatment, such as where a partial or total joint replacement is desired. Advantageously, the SC™ treatment may be provided as a first or initial treatment, reserving for the future and possibly forestalling until a later date than otherwise might be the case more invasive treatments such as partial or total joint replacement.

Various surgical treatments to address subchondral defects known as bone marrow lesions have previously been attempted. Between May and November 2008, three (3) surgeries were performed at Riddle Hospital in Media, Pa. in the United States. On May 12, 2008, Dr. Peter F. Sharkey performed a right knee arthroscopy with arthroscopically assisted stabilization of a patient's right knee with a medial tibial plateau fracture. During the procedure, a cannulated bone biopsy needle was placed into the bone under fluoroscopic guidance, and augmentation material was injected. The injected augmentation material was Stryker Orthopedics Hydroset (Bone Substitute Material). The surgeon expressed difficulty in injecting the bone substitute material.

On Oct. 27, 2008, Dr. Steven B. Cohen performed a left knee arthroscopy, partial medial meniscectomy, drilling of osteochondral lesion using retrograde technique, and debridement chondroplasty of patellofemoral chondrosis on a patient's left knee with medial meniscus tear and left knee osteochondral defect with bone marrow lesion of the medial femoral condyle. During the procedure, an Anterior Cruciate Ligament (ACL) portal-creation device was repurposed for this surgery. The tibial probe was placed on the medial femoral condyle, with the tunnel guide secured proximally on the thigh. The surgeon expressed difficulty in positioning and stabilizing the guide. A cannulated pin was placed through the tunnel guide and placed distally into the medial femoral condyle. No implantable material was injected into the bone in this case.

On Nov. 10, 2008, Dr. Steven B. Cohen performed a right knee arthroscopic-assisted repair of a tibial plateau fracture bone marrow lesion with subchondral fracture using bone cement, partial medial and partial lateral meniscectomy to treat medial meniscus tear, and arthroscopic debridement and chondroplasty of medial, lateral, and patellofemoral compartments to treat compartment chondrosis. During the procedure, a guide pin was inserted into the medial tibial plateau, and an endo button drill bit was used to expand the drill hole. One (1) cubic centimeter (cc) of cement was inserted into the bone. A second drill hole was made from below, and a second cubic centimeter (cc) of cement was inserted into the bone.

The experiences gained from these previous surgeries helped to develop the fundamental theories underlying the SUBCHONDROPLASTY™ procedure and the number of treatment modalities, associated devices, instruments and related methods of use for performing the SUBCHONDROPLASTY™ procedure, which are disclosed in the aforementioned publications. These treatment modalities may be used alone or in combination, as will be described in detail below.

In one treatment modality, the subchondral bone in the region of the bone marrow lesion or defect can be strengthened by introduction of a hardening material, such as a bone substitute, at the site. The bone substitute may be an injectable calcium phosphate ensconced in an optimized carrier material. In an SC™ procedure, the injected material may also serve as a bone stimulator that reinvigorates the desired acute bone healing activity.

For example, polymethylmethacrylate (PMMA) or calcium phosphate (CaP) cement injections can be made at the defect site. PMMA injection may increase the mechanical strength of the bone, allowing it to withstand greater mechanical stresses. CaP cement injection may also increase the mechanical strength of the bone, while also stimulating the localized region for bone fracture repair. In one embodiment, the injection can be made parallel to the joint surface. In another embodiment, the injection can be made at an angle to the joint surface. In yet another embodiment, the injection can be made below a bone marrow lesion. Preferably, the injection is made without disrupting the joint surface.

In another treatment modality, the subchondral bone region can be stimulated to trigger or improve the body's natural healing process. For example, in one embodiment of this treatment modality, one or more small holes may be drilled at the region of the defect to increase stimulation (e.g., blood flow, cellular turnover, etc.) and initiate a healing response leading to bone repair. In another embodiment, after holes are drilled an osteogenic, osteoinductive, or osteoconductive agent may be introduced to the site. Bone graft material, for example, may be used to fill the hole. This treatment modality may create a better load-supporting environment leading to long term healing. Electrical or heat stimulation may also be employed to stimulate the healing process of a chronically injured bone. Chemical, biochemical and/or biological stimulation may also be employed in an SC™ procedure. For instance, stimulation of bone tissue in an SC™ procedure may be enhanced via the use of cytokines and other cell signaling agents to trigger osteogenesis, chondrogenesis, and/or angiogenesis to perhaps reverse progression of osteoarthritis.

In yet another treatment modality, an implantable device may be implanted into the subchondral bone to provide mechanical support to the damaged or affected bone region, such as where an insufficiency fracture or stress fracture has occurred. The implant may help create a better load distribution in the subchondral region. In the knees, the implant may support tibio-femoral compressive loads. In addition, the implant may mechanically integrate sclerotic bone with the surrounding healthy bone tissue. The implants may be place in cancellous bone, through sclerotic bone, or under sclerotic bone at the affected bone region. The implant may also be configured as a bi-cortical bone implant. In one embodiment, one side of the implant can be anchored to the peripheral cortex to create a cantilever beam support (i.e., a portion of the implant is inserted into bone but the second end stays outside or near the outer surface of the bone). The implant may be inserted using a guide wire. In one example, the implant may be inserted over a guide wire. In another example, the implant may be delivered through a guide instrument.

The implant may further be augmented with a PMMA or CaP cement injection, other biologic agent, or an osteoconductive, osteoinductive and/or osteogenic agent. The augmentation material may be introduced through the implant, around the implant, and/or apart from the implant but at the affected bone region, such as into the lower region of a bone marrow lesion or below the lesion. For example, the implant may act as a portal to inject the augmentation material into the subchondral bone region.

While each of the above-mentioned treatment modalities may be administered independent of one another, it is contemplated that any combination of these modalities may be applied together and in any order so desired, depending on the severity or stage of development of the bone defect(s). Suitable implantable fixation devices for the surgical treatment of these altered bone regions or bone defects, especially at the subchondral level, are disclosed in co-pending and co-owned U.S. Patent Application Publication No. 2011/0125265 entitled “IMPLANTABLE DEVICES FOR SUBCHONDRAL TREATMENT OF JOINT PAIN,” U.S. Patent Application Publication No. 2011/0125264 entitled “IMPLANTABLE DEVICES FOR SUBCHONDRAL TREATMENT OF JOINT PAIN,” and U.S. Patent Application Publication No. 2011/0125272 entitled “BONE-DERIVED IMPLANTABLE DEVICES FOR SUBCHONDRAL TREATMENT OF JOINT PAIN,” all of which were filed on Nov. 19, 2010, the contents of which are herein incorporated in their entirety by reference. These devices and instruments can be use in combination with cements or hardening materials commonly used to repair damaged bone by their introduction into or near the site of damage, either to create a binding agent, cellular scaffold or mechanical scaffold for immobilization, regeneration or remodeling of the bone tissue. As previously stated, treatment of the bone defect at the subchondral level preferably is performed without disrupting the joint surface.

In a healthy joint such as a tibio-femoral joint, the compressive load between the contact bones (i.e., the femur and the tibia) is properly distributed, thus keeping the contact stresses in the cartilage to a reasonably low level. As the cartilage starts to wear out or degenerate locally, the tibio-femoral contact area reduces and starts to get localized at the site of the cartilage defect. The localization of the stresses may also occur due to varus or valgus deformity. Sometimes, the condition may occur because of osteoporosis, where bone becomes weak and is no longer able to support normal loads. This condition leads to higher localized contact stresses in the cartilage, and the subchondral region below the cartilage. Once the stresses reach beyond a certain threshold level, it leads to defects like bone marrow lesions and edema, and perhaps generates knee pain. If the problem persists, the high contact stresses can lead to sclerotic bone formation as well. The presence of sclerotic bone can compromise vascularization of the local area, and also create a mechanical mismatch in the bone tissue. This mismatch may start to expedite degeneration of all parts of the joint leading to increased levels of osteoarthritis.

Pain associated with osteoarthritic joints can be correlated to bone defects or changes at the subchondral level. In particular, bone defects such as bone marrow lesions, edema, fissures, fractures, etc. near the joint surface lead to abnormal stress distribution in the periarticular bone, which may or may not cause inflammation and generate pain. By altering the makeup of the periarticular bone (which may or may not be sclerotic) in relation to the surrounding region, it is possible to change the structural integrity of the affected bone, leading to a resolution of the inflammation. Treatment of the bone in an effort to alter the structural makeup of the affected periarticular bone leads to reduced inflammation and pain has proven to be successful. Over time, restoration of normal physiologic stress distribution can be achieved in load bearing joints such as the hip and knee, and mechanical congruity restored, thereby resulting in healing of the inflammation and reduction or elimination of pain.

In general, the present disclosure provides embodiments related to devices, instruments and associated methods for the surgical treatment of a joint, and particularly a subchondral bone defect at that joint region. More specifically, the embodiments relate to methods of treating a bone defect of a joint at the subchondral level with implants derived from bone, and associated delivery instruments. These instruments and devices may be used in a manner consistent with the subchondral procedures previously described, and may optionally contain depth control features for more accuracy.

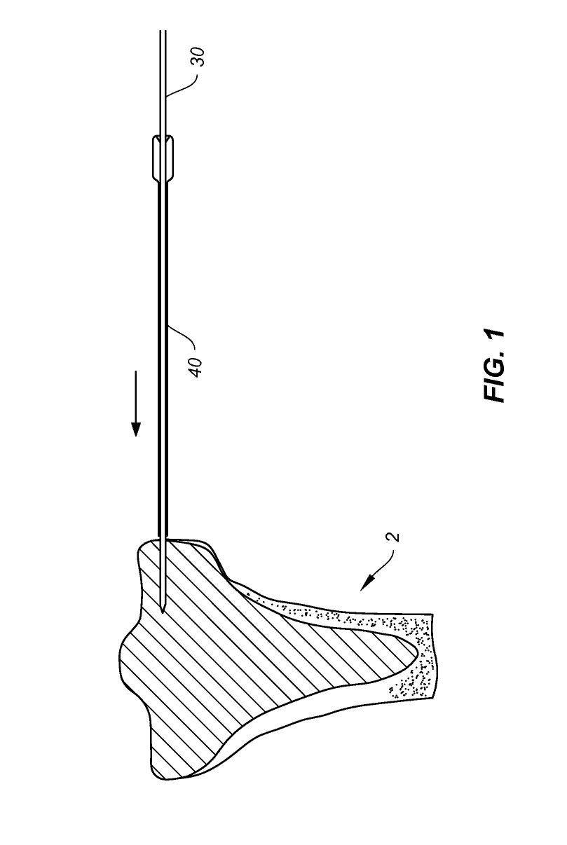

Turning now to the drawings, FIGS. 1, 2A, 2B, and 3-6 illustrate an exemplary embodiment of a method of treating a subchondral bone defect of a joint in which FIG. 1 shows a step of treating a bone defect, such as a bone marrow lesion or bone marrow edema, at the subchondral level of bone 2. In the illustrated embodiment, the bone 2 may be a tibia of a knee joint. As shown, a wire 30 may be drilled to the subchondral area to be treated in the bone 2 through a wire sleeve 40, along the direction shown by the arrow. The wire sleeve 40 may serve to protect as well as stiffen the wire 30 and keep it from skiving as it is drilled into the bone 2, particularly in the case of cortical bone. The wire sleeve 40 could be positioned by using a navigation guide and may be used in accordance with templates or other imaging tools to target the defect. Preferably, the wire may be drilled straight through the cortical bone and into the cancellous bone of the tibia 2. The depth of the wire may be equivalent to an expected delivery pin drilling depth.

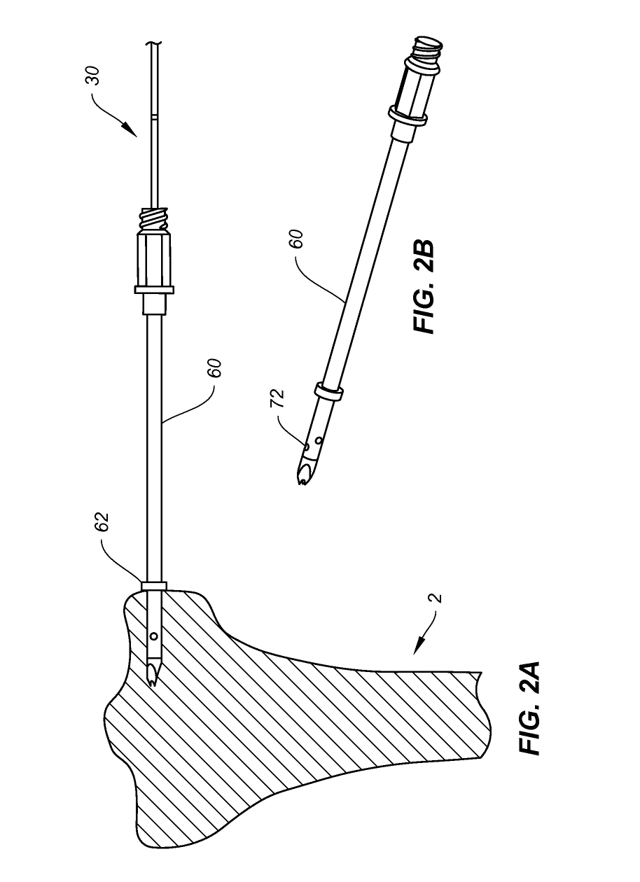

FIG. 2A shows another step in the exemplary treatment method in which the wire 30 may remain after the wire sleeve 40 has been removed. A delivery pin or device 60 for the injection of treatment materials such as those described above may be drilled over the wire 30 and into the bone. As further shown in FIG. 2B, the delivery device 60 may comprise fenestrations 72 and be fully cannulated. In addition, the delivery device 60 may have an optional flange or collar 62 that controls the depth to which the device 60 is drilled into the bone 2. Other visual markers may also be used for this purpose, such as fluoroscopic markers (i.e., radiopaque markers) or visual markers like etchings or colored bands. The drilling depth of the pin 60 may be the length of the intended implant 20, in one instance.

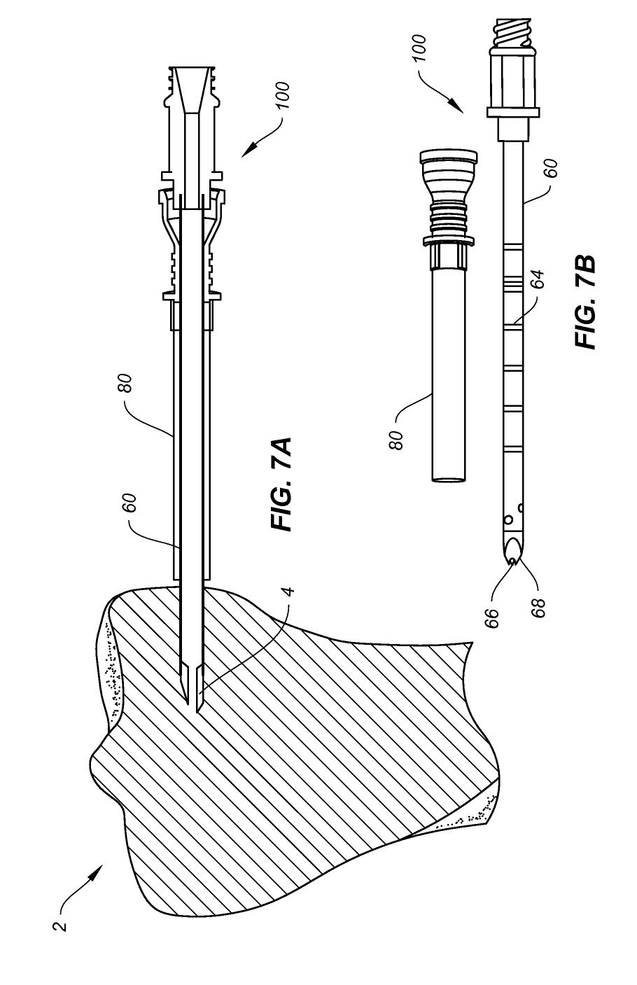

In addition to the collar 62, the delivery pin or delivery device 60 may also utilize other features or components to control its drill depth. As shown in FIG. 7B, visual or radiolucent (i.e., fluoroscopic) markers 64 may be provided along the shaft of the delivery device 60 to assist the user in determining depth of insertion. Another optional component is a depth gauge or sleeve 80 that slides over the delivery device 60 such that the delivery device 60 fits securely into the gauge 80 to a certain predetermined depth, forming a delivery instrument 100 as shown. Once assembled with the delivery device 60 inside the depth gauge 80, the delivery instrument 100 would only protrude to a certain length before the gauge 80 stops any further advancement into the bone 2.

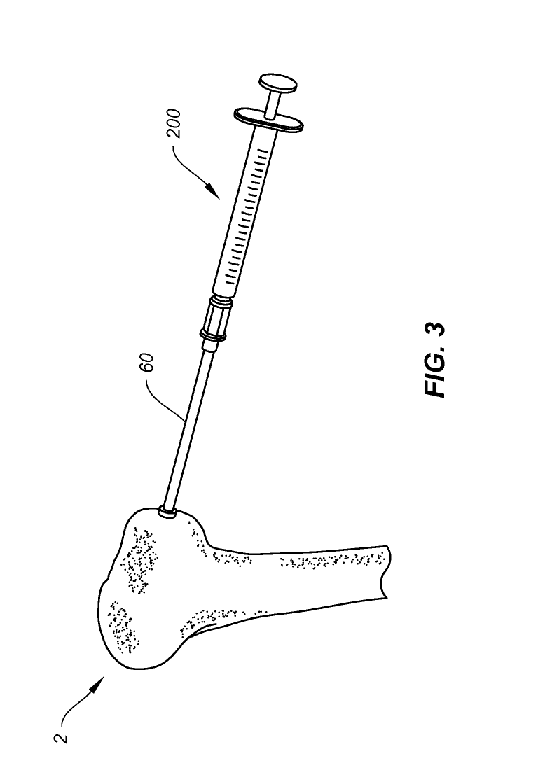

FIG. 3 shows another step in the treatment method in which the wire 30 has been removed, and an injection system 200 is attached to the delivery device 60. In the present embodiment, the injection system 200 may be a syringe. However, other injection systems may be employed, of course. The injection system 200 may house an injectable material that will stimulate healing, bone regrowth, support the joint, or otherwise mitigate pain. Suitable injectable treatment materials are described above in reference to SC™ treatment modalities. After injection of the material to the area to be treated, the injection system 200 may be removed.

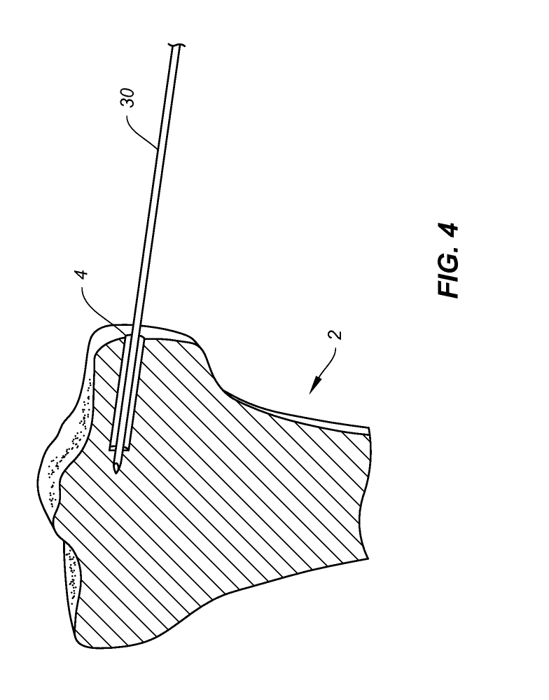

FIG. 4 shows still another step in the treatment method in which the wire 30 and wire sleeve 40 are reintroduced to the cavity 4 in the area of the bone 2 to be treated. As shown, the wire 30 may be drilled back into the bone 2 using the wire sleeve 40 to control its depth into the bone 2. The wire 30 and the wire sleeve 40 may be pushed through the delivery device 60, forcing any residual injectable material within the delivery device 60 out of fenestrations 72 and into the bone 2. The wire 30 can be drilled an additional amount to anchor it to the bone 2. Afterwards the wire sleeve 40 and delivery device 60 may both be removed, leaving the wire 30 and a cavity 4 or void created by the previous steps.

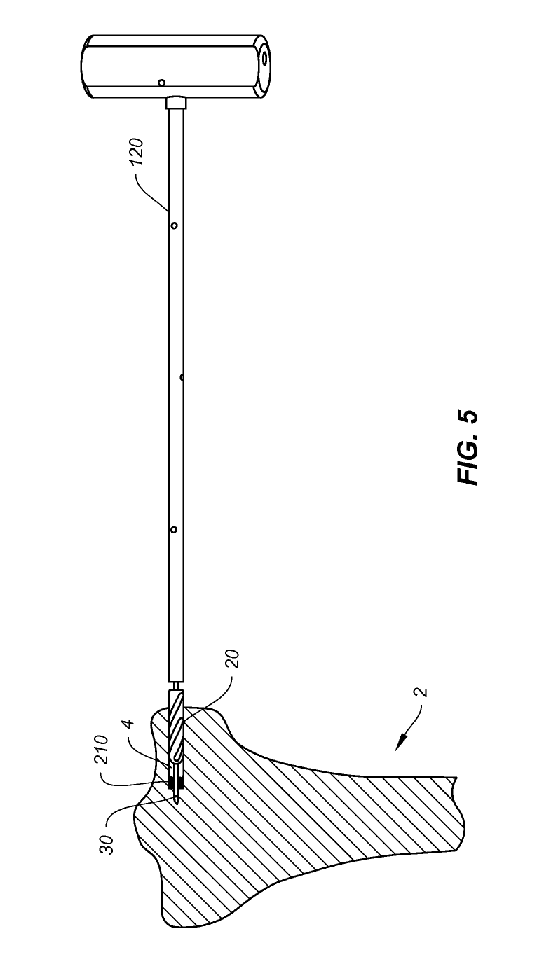

FIG. 5 shows yet another step in the treatment method in which an implant 20 is inserted into the cavity. In the present embodiment, the implant 20 is a bone-derived implant such as an allograft. Additionally, the implant 20 is fully cannulated. As shown, the cannulated implant 20 may be slid over the anchored wire 30 up to the skin or bone 2. An inserter instrument such as a pushing instrument 120 similar to the one shown may be provided to urge the implant 20 down the wire 30 and into the cancellous bone 2. The cannulated implant 20 should both fill the bone void or cavity 4 and also act as a plug for the injected material 210.

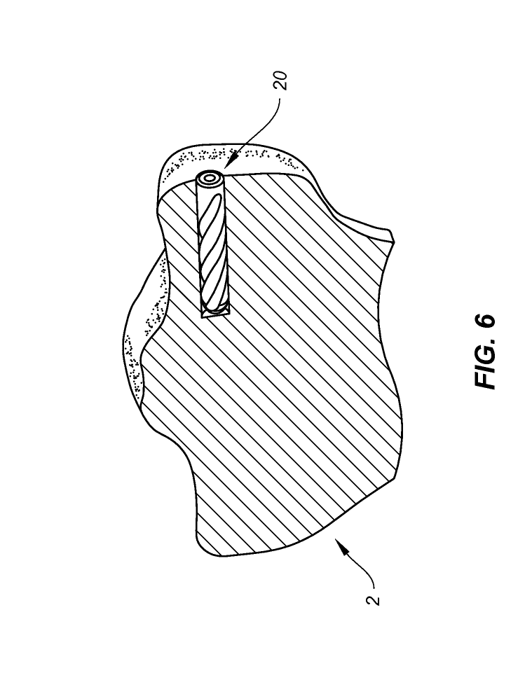

Finally, as shown in FIG. 6, once the implant 20 is sufficiently seated within the cavity 4, the wire 30 may be removed from the bone 2. At this stage, the implant 20 should be flush or pressed firmly into the cortical bone 2. The incision may then be sealed.

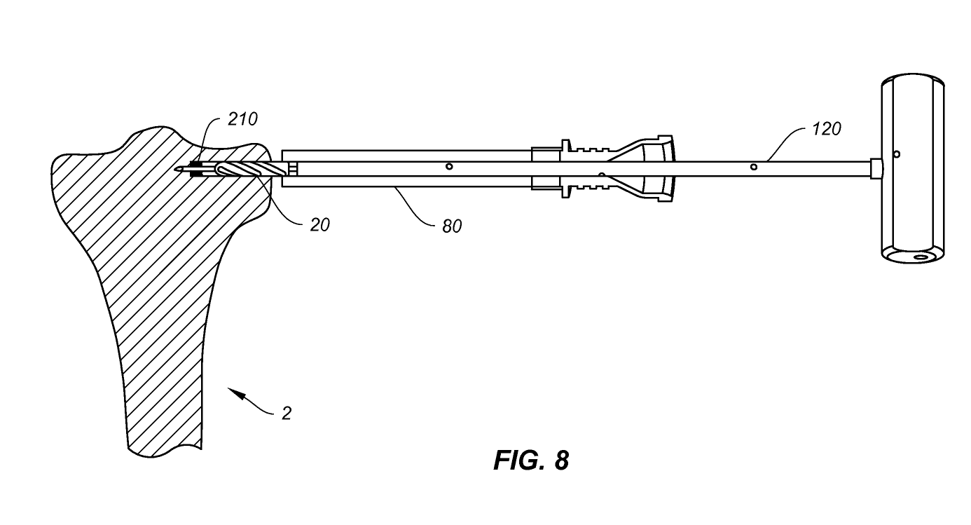

FIGS. 7A, 7B and 8 illustrate another exemplary embodiment of a method of treating a subchondral bone defect of a joint without the need for a wire 30. As shown, a cavity or void may be created in the bone 2 with the delivery device 60. Delivery device 60 may optionally include a bone cutting edge 66 at the insertion tip 68 that cuts into bone tissue. Accordingly, the delivery device 60 may be inserted into bone 2 by exerting pressure and forcing the delivery device 60 into the bone 2, to core out a cavity or bone void 4. The delivery device 60 may be used in combination with a depth gauge or protective sleeve 80 to create a delivery instrument 100 that allows both cavity creation and injection of augmentation or bone hardening material 210.

As shown in FIG. 7A, the delivery device 60 may be slid through the protective sleeve or depth gauge 80. The depth gauge 80 provides an added level of strength to the process during the cavity creation step, and also protects material from extruding out of the cavity 4 during the injection process. Once the augmentation material 210 has been injected, the delivery device 60 may be pulled out of the sleeve 80, and a solid implant 20 may be inserted to plug the cavity 4. The protective sleeve 80 may be used again to direct the implant 20 into the cavity 4 with an inserter instrument 120 such as pushing instrument or impacter. The implant 20 may be cannulated, similar to the implant of FIGS. 5 and 6, or may be solid. In addition, the implant 20 may comprise bone-derived material and may be, for example, an allograft implant.

It is understood that other forms of implantable devices and variations of the implant 20 may be utilized in the methods of the present disclosure. For example, suitable implants are also disclosed in co-pending and co-owned U.S. Patent Application Publication No. 2011/0125265, filed Nov. 19, 2010 and entitled “IMPLANTABLE DEVICES FOR SUBCHONDRAL TREATMENT OF JOINT PAIN,” U.S. Patent Application Publication No. 2011/0125264, filed Nov. 19, 2010 and entitled “IMPLANTABLE DEVICES FOR SUBCHONDRAL TREATMENT OF JOINT PAIN,” and U.S. Patent Application Publication No. 2011/0125272, filed Nov. 19, 2010 and entitle “BONE-DERIVED IMPLANTABLE DEVICES FOR SUBCHONDRAL TREATMENT OF JOINT PAIN,” the contents of which are herein incorporated in their entirety by reference.

As previously mentioned, a navigation guide, template guide, or other imaging tool may be provided with the systems of the present disclosure to guide the instrument, device or implant toward the subchondral defect or bone cavity. Such navigation or imaging tools or guides may be used to ascertain a desired access path for targeting the location of the subchondral region near the subchondral defect to be treated. In one example, this access path may be determined using a mapping system that provides a set of coordinates for targeting the location of the subchondral region. Such a mapping system may be similar to the one disclosed in co-pending and co-owned U.S. Patent Application Publication No. 2011/0125201, filed Nov. 19, 2010 and entitled “COORDINATE MAPPING SYSTEM FOR JOINT TREATMENT,” the contents of which are herein incorporated in their entirety by reference.

In addition to the mapping system described above, other navigation or imaging tools suitable for use with the systems and methods of the present disclosure may include those disclosed in co-pending and co-owned U.S. Patent Application Publication No. 2011/0125159, filed Nov. 19, 2010 and entitled “INSTRUMENTS FOR A VARIABLE ANGLE APPROACH TO A JOINT,” U.S. Patent Application Publication No. 2011/0125200, filed Nov. 19, 2010 and entitled “NAVIGATION AND POSITIONING INSTRUMENTS FOR JOINT REPAIR AND METHODS OF USE,” U.S. Patent Application Publication No. 2012/0245645, filed Feb. 22, 2012 and entitled “NAVIGATION AND POSITIONING SYSTEMS AND GUIDE INSTRUMENTS FOR JOINT REPAIR,” and U.S. patent application Ser. No. 14/022,001 filed on Sep. 9, 2013 and entitled “NAVIGATION INSTRUMENTS FOR SUBCHONDRAL BONE TREATMENT,” the contents of which are herein incorporated in their entirety by reference.

Finally, the delivery device 60 of the present disclosure may be similar to those disclosed in co-pending and co-owned U.S. Patent Application Publication No. 2012/0316513, filed Jun. 8, 2012 and entitled “INSTRUMENTS AND DEVICES FOR SUBCHONDRAL JOINT REPAIR,” and further include adapter and components as described in this application. Likewise, as mentioned above, the delivery device 60 may incorporate various depth control features or components. Additional depth control features or components that may be incorporated into the systems and methods of the present disclosure are found in U.S. patent application Ser. No. 14/021,785 filed on Sep. 9, 2013 and entitled “INSTRUMENTS FOR CONTROLLED DELIVERY OF INJECTABLE MATERIALS INTO BONE.” The contents of both these applications are herein incorporated in their entirety by reference.

Other embodiments will be apparent to those skilled in the art from consideration of the specification and practice of the embodiment disclosed herein. It is intended that the specification and examples be considered as exemplary only, with a true scope and spirit of the embodiment being indicated by the following claims.

What is claimed is:

1. A system for treating a subchondral defect of a bone of a joint, comprising:
a delivery instrument for injecting a bone augmentation material, the instrument comprising a fenestrated delivery device configured to attach to an injection system, the delivery device having a bone cutting edge at its tip to create a bone cavity in the bone, and a protective sleeve configured to receive the fenestrated delivery device;
an injection system containing bone augmentation material; and
an implant comprising bone derived material, the implant being configured to plug the bone cavity.

2. The system of claim 1, further comprising a guide wire.

3. The system of claim 1, wherein the implant is cannulated.

4. The system of claim 1, wherein the delivery device comprises a collar for depth control.

5. The system of claim 1, wherein the delivery device comprises visual markers for depth control.

6. The system of claim 1, further including an impacter instrument to push the implant through the protective sleeve and into the bone cavity.

7. The system of claim 1, wherein the bone augmentation material comprises a bone hardening or bone substitute material.

8. The system of claim 1, wherein the implant is an allograft.

9. The system of claim 1, further comprising a navigation guide, template guide, or other imaging tool.

10. A method of treating a subchondral defect of a bone, comprising:
inserting a guide wire into the bone toward the subchondral defect, the guide wire being inserted without violating the articular surface of the bone;
inserting a delivery device over the guide wire and into the bone to create a bone cavity at a subchondral area near the subchondral defect;
injecting a bone augmentation material into the bone cavity; and
inserting an implant into the bone cavity;
wherein the method preserves the articular surface of the bone.

11. The method of claim 10, wherein the wire is inserted using a navigation guide, template guide, or other imaging tool.

12. The method of claim 10, wherein the injectable material comprises a bone substitute or bone hardening material.

13. The method of claim 10, wherein the subchondral defect comprises a bone marrow lesion or bone marrow edema.

14. The method of claim 10, further including inserting the delivery device through a protective sleeve and into the bone.

15. The method of claim 10, further including pushing the implant through a protective sleeve into the bone cavity using an impacter instrument.

16. A method of treating a subchondral defect of a bone, comprising:
inserting a delivery device through a protective sleeve and toward the subchondral defect to create a bone cavity at a subchondral area near the subchondral defect, the delivery device being inserted without violating the articular surface of the bone;
injecting a bone augmentation material into the bone cavity; and
inserting an implant into the bone cavity;
wherein the method preserves the articular surface of the bone.

17. The method of claim 16, wherein the wire is inserted using a navigation guide, template guide, or other imaging tool.

18. The method of claim 16, wherein the injectable material comprises a bone substitute or bone hardening material.

19. The method of claim 16, wherein the subchondral defect comprises a bone marrow lesion or bone marrow edema.

20. The method of claim 16, wherein the implant is inserted by pushing the implant through the protective sleeve into the bone cavity using an impacter instrument.
